(19) United States
(12) Patent Application Publication
(10) Pub. No.: US 2004/0143854 A1
(43) Pub. Date: Jul. 22, 2004

(54) Method for generating a genetically modified organism

(75) Inventors: Bert Klebl, Gunzelhofen (DE); Anja Stadler, Furstenfeldbruck (DE); Rosemarie Sollner, Munchen (DE); Ekkherd Leberer, Germering (DE); Almut Nitsche, Wiesbaden (DE)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: Aventis Pharma Deutschland GmbH, Frankfurt am Main (DE)

(21) Appl. No.: 10/736,801

(22) Filed: Dec. 16, 2003

Related U.S. Application Data
(60) Provisional application No. 60/466,400, filed on Apr. 29, 2003.

(30) Foreign Application Priority Data
Dec. 17, 2002 (DE) 10258885.6-41

(51) Int. Cl. A01K 67/027

(52) U.S. Cl. 800/3; 800/14

(57) ABSTRACT

A method for generating a genetically modified organism for drug screening is presented in which heterologous expression of at least one protein in caused in the organism followed by analysis of the modified gene expression pattern of the expressing organism and phenotyping by altering the expression of the compensating differentially regulated genes. Assays using the genetically modified organism are also presented.

The invention relates to a method for generating a nonhuman, genetically modified organism for drug screening and to assays based on such organisms.

Genetically modified yeasts which express heterologously the target protein which is to be inhibited by the substance to be tested are known to be used for drug screening. Heterologous expression means, within the scope of the present invention, expression of a gene foreign to the organism or expression of a gene endogenous to the organism with an altered expression pattern, in particular enhanced or reduced expression and/or an expression which is altered with respect to time and/or space (e.g. other compartments, in higher organisms other tissues, for example). In the simplest case, heterologous expression leads to a detectable modified phenotype, usually a growth inhibition, of the yeast. Growth inhibition means, within the scope of the present invention, a reduced rate of proliferation and/or a reduced growth in size and also includes cell death (apoptotic or necrotic). The type of growth inhibition occurring also depends on the organism; thus, in yeasts either a proliferation arrest or a lysis can be observed, whereas in eukaryotic cells which are originally derived from multicellular organisms apoptosis can also sometimes be observed. If heterologous expression results in a modification of the behavior and/or the morphology of the organism, which is perceptible from the outside (i.e. a modified phenotype), the genetically modified organism can readily be used for drug screening, the efficacy of the substances tested being determinable on the basis of their ability to eliminate or reduce the phenotype (e.g. growth inhibition). In the example of the yeast system with growth inhibition as modified phenotype, this is preferably carried out by simple growth assays which are also suitable for high throughput screening (HTS). Any alteration, perceptible from the outside, of the genetically modified organism (shape, size, etc.) or of its behavior (growth, rate of cell division, etc.) in comparison with the genetically unmodified organism or with the organism which does not express the heterologous protein(s) or protein fragment(s) is referred to as modified phenotype. Phenotyping thus refers to causing such a modification.

However, this method of the prior art has the disadvantage of only a small proportion of heterologously expressed genes producing a phenotype of the genetically modified organism, which is usable for drug screening. Thus it is assumed that, for example, only approx. 20-30% of all heterologously expressed kinases cause a growth inhibition in the yeast, which can be utilized for drug screening. In the case of the remaining 70-80%, growth inhibition is so low that it cannot be used for screening (too small a difference in comparison with the control leads to a high background and thus to too large a number of false positives) or it is not present at all.

There exists, therefore, the need for a method for generating a genetically engineered organism for drug screening which does not have the disadvantages of the prior art and is, in particular, suitable for making accessible to drug screening also those heterologously expressed genes which do not produce any phenotype or any phenotype usable for screening, in particular for HTS, in the organism in which they are heterologously expressed.

According to the invention, this object is achieved by a method for generating a genetically modified organism for drug screening, which comprises the steps

a) causing heterologous expression of at least one protein or protein fragment by genetic modification of the organism.

b) preferably, this is followed by determining the phenotype of the genetically modified organism.

c) analyzing the modified gene expression pattern and identifying compensating differentially regulated genes.

d) phenotyping the organism (preferably by deletion, mutagenesis or overexpression of the compensatingly regulated genes to enhance or generate a phenotype in combination with the heterologously expressed protein or protein fragment).

The invention is based on the finding by the inventors that the lack of a detectable phenotype for heterologous expression of most genes is based on the fact that the genetically modified organism up- or downregulates (i.e. compensatingly differentially regulates) the expression of some genes as response to expression of the heterologously expressed protein or protein fragment. Differentially regulated means, in this case, regulated differently than in the genetically modified organism or without heterologous expression of the heterologously expressed protein or protein fragment. Compensatingly means that that differential gene regulation is a response to heterologous expression of the protein or protein fragment.

The invention makes possible the development of a platform technology in a cellular model, preferably the yeast, in contrast to the simple biochemical model. Using the assay system it is possible, for example, to identify inhibitors from chemical libraries, from CombiChem libraries and from extracts of natural substances. The assay system can be adapted to 96-, 384- or 1 536-well plates or to other formats common for cellular assays. The format to be chosen depends partly also on the chosen organism, the selection being within the ability of the skilled worker.

The method of the invention is particularly suitable for genes and proteins or protein fragments whose heterologous expression in the desired organism does not result in any detectable modification of the phenotype in comparison with the genetically unmodified organism or the organism which does not heterologously express said protein or protein fragment. It is possible, for example, to assay protein kinases as well as other gene products which cause a transcriptional response. It may, however, also be applied to a detectably modified phenotype, in particular if a modified phenotype, although detectable, is not suitable or not appropriate for the use in drug screening, due to particular reasons. Said phenotype may be enhanced by phenotyping or modified in such a way that it can be used for drug screening. Accordingly, phenotyping refers, within the scope of the present invention, to causing or enhancing a phenotype in the genetically modified organism expressing heterologously the protein(s) or protein fragment(s), which phenotype can be distinguished from the organism which does not heterologously express the protein(s) or protein fragment(s) or from the genetically unmodified organism.

Suitable organisms are preferably cells, here eukaryotic as well as prokaryotic cells, or else multicellular nonhuman organisms which are suitable for drug screening, for example Drosophila and preferably C. elegans. Suitable eukaryotic cells are preferably cultured cell lines which were originally obtained from multicellular organisms, for example 3T3, CHO, HeLa, or else other or eukaryotic unicellular organisms, in particular yeasts. Particularly suitable among the yeasts are, in turn, those of the strains S. cerevisiae or S. pombe. Suitable laboratory strains of yeast cells or suitable eukaryotic cell lines are sufficiently well known to the skilled worker.

Suitable proteins and protein fragments are in principle all those whose heterologous expression in the organism results in an alteration of the expression pattern of endogenous genes. Advantageous are all proteins and protein fragments which are of interest with respect to finding new active substances, with kinases, phosphatases, GPCRs, (in particular small) GTPases, proteases and ion channels being particularly preferred within the scope of the present invention.

The term drug screening comprises, within the scope of the present invention, any type of search for substances which act on the activity of one or more particular target genes and/or target proteins, using at least one genetically modified organism. In principle, any types of substances are suitable here, for example any types of natural substances (i.e. molecules occurring in nature, in particular biomolecules) as well as not naturally occurring, synthetically produced chemicals and substances/derivatives derived from natural substances, in particular biological molecules (e.g. modified peptides or oligonucleotides).

Heterologous expression may comprise the introduction of a foreign gene or else the modified expression of a gene endogenous to the organism, for example by introducing an appropriate expression vector. The genetic modification required therefor may concern the modification of the genome of the organism (e.g. by means of stable vectors integrating into the genome or by various types of mutagenesis), may be episomal or may comprise simply the introduction of suitable vectors which require constant selection by means of one or more selection markers in order to remain in the organism. The most suitable type depends on various factors, inter alia also on the type of organism, and can be readily determined by the competent skilled worker.

The heterologous expression relates to at least one protein or protein fragment but may also relate to a plurality of proteins or protein fragments. It may be expedient to verify expression of the heterologous protein/fragment by suitable methods (PCR, Northern blot, Western blot, etc.), before the gene expression pattern of the genetically modified organism is compared, and thus analyzed, with the organism lacking expression of said heterologous protein. The analysis is carried out by suitable measures which are sufficiently well known to the skilled worker, the use of array (preferably DNA/RNA or protein microarrays) or chip systems being particularly suitable for this purpose. By comparing the expression patterns of a control organism (e.g. a wild-type organism or an organism into which merely the empty vector has been introduced or, for inducible systems, the genetically modified organism in which expression of the heterologous gene has not been induced) and of the genetically modified organism expressing the heterologous gene. Such gene products which appear at all/to an increased/reduced extent or not at all in the expression pattern of the genetically modified organism expressing the heterologous gene in contrast to the expression pattern of the control organism are thus regarded as compensatingly differentially regulated genes and may be used for phenotyping said genetically modified organism.

Phenotyping refers to causing or enhancing a phenotype distinguishable from the wild-type organism in the genetically modified organism (or, for inducible systems, a phenotype which is only produced by the genetically modified organism with heterologous expression of the protein(s) or protein fragment(s) and which is not produced in the noninduced state of said organism, when the protein(s) or protein fragment(s) are not expressed), with the phenotype being preferably suitable for evaluation in HTS drug screening. Said causing or enhancing may take place here, for example, on reducing or eliminating expression of one or more compensatingly upregulated genes (this may be carried out, for example, by genomic knock out of one or more of the compensatingly differentially regulated genes or by mutagenesis) or enhanced expression of one or more compensatingly downregulated genes (this may be carried out, for example, by heterologous expression of one or more compensatingly differentially downregulated genes, using suitable expression vectors). In this way it is possible to produce a phenotype, endogenous to the organism and caused by the heterologously expressed gene, which phenotype has been prevented due to compensatingly differential regulation of one or more genes (preferably growth inhibition, but, in particular in multicellular organisms, other phenotypes are also possible here).

Another possibility is also to label one or more compensatingly upregulated genes by means of a suitable marker/tag (which is coupled to the gene product, for example) or by means of a reporter which is under the control of the enhancer and/or promoter of the compensatingly upregulated gene and which is introduced into the organism. Suitable reporters are known to the skilled worker, and suitable here are, in particular, any types of luminescent proteins (e.g. GFP, BFP, etc.) or else other reporters capable of generating a detectable signal (e.g. luciferase, β-galactosidase) and growth markers for auxotrophic strains such as, for example, HIS3, URA3, LEU2, TRP1, and antibiotic resistance genes such as, for example, for kanamycin or G418. Other types of phenotyping are also conceivable.

Following phenotyping, it is expedient to check the success of said phenotyping by suitable methods (e.g. measuring the rate of proliferation, cell counting or determination of size or morphology, etc. and comparison with the phenotype of heterologous expression not taking place).

According to a preferred form of carrying out the method of the invention, phenotyping is carried out by means of deletion, mutagenesis or overexpression of at least one compensatingly regulated gene.

According to a preferred embodiment, phenotyping is carried out by reducing/eliminating the compensatingly differential expression or by labeling at least one compensatingly differentially regulated gene.

In this connection, heterologous expression may result in compensatory up- and also downregulation of at least one gene endogenous to the organism but may also result in one or more genes being upregulated and one or more other genes being downregulated.

It is also particularly convenient if heterologous expression of the protein or protein fragment is inducible. Suitable systems are known to the competent skilled worker, suitable examples thus being galactose- or copper-regulated promoters, the Tet-On Tet-Off system, etc. This may involve either inducibly switching on expression of a gene foreign or endogenous to the organism (inducible knock in) or inducibly reducing or completely switching off expression of a gene endogenous to the organism (inducible knock out). To this end, the genetic modification expediently comprises introducing a vector enabling inducible expression of the protein or protein fragment, preferably one with galactose-(GAL1/GAL10) or copper-(CUP1) regulated promoters, tetracycline-inducible vector or tissue-specifically inducible promoters such as, for example, hsp 16-2, unc-119, unc-54, mec-7, or myo-3 in C. elegans.

According to a preferred embodiment, the organism is C. elegans, a prokaryotic or eukaryotic cell and, particularly preferably, a yeast cell, preferably a yeast cell of the strain S. cerevisiae.

The modified gene expression is preferably analyzed by DNA/RNA profiling with the aid of cDNA or oligonucleotide microarrays, but the analysis may in principle include any modifications of the mRNA or protein steady state (transcription, translation, stabilization, etc.) and thus may also be carried out by protein profiling as well as with the aid [lacuna] protein arrays.

In an advantageous design of the method, phenotyping is carried out by reducing or eliminating the compensatingly differential regulation. If the compensatingly differentially regulated gene is expressed stronger than in control organisms, said reduction or elimination is carried out by completely or partially inhibiting the enhanced expression. This is preferably carried out by crossing with a deletion strain and subsequent selection of the double mutants (particularly suitable when the organism is yeast), by genomic knock out using suitable vectors (these are known to the skilled worker and likewise very suitable in yeasts, here especially Saccharomyces cerevisiae), mutagenesis by radiation and/or mutagenic substances or introduction of antisense vectors or the like which inhibit protein production of the gene in question. To this end, it is particularly advantageous if the knock out of the compensatingly differentially regulated gene comprises the knock in of a reporter gene such as, for example, β-galactosidase, luciferase or growth markers such as HIS3, ADE2, URA3 or resistance markers such as, for example, for kanamycin. The reporter gene may then be used as signal in the subsequent assay to detect and quantify the efficacy of the drugs to be tested. This involves preferably replacing at least part of the coding sequence of the differentially regulated gene with the coding sequence (also including parts of said sequence which are sufficient for being detectable) of a reporter gene (e.g. luciferase, β-galactosidase, etc.). If the compensatingly differentially regulated gene is less strongly expressed than in the control organism, reduction or elimination is effected by enhancing expression, preferably by crossing-in, introducing an episomal or another expression vector capable of selection or by genomic knock in (the methods above are particularly suitable for using yeast as organism). Preferably, reducing or eliminating the compensatingly differential regulation results in a growth inhibition of the genetically modified organism, but other phenotypes may also be advantageous.

Another aspect of the invention relates to a genetically modified, phenotyped organism generated by the method of the invention.

In particular, the invention relates to a genetically modified organism having genetically modified expression of at least one endogenous or foreign gene, which expression results in the compensatingly differential regulation of at least one other gene endogenous to said organism and thus preferably stops or inhibits an assessable/detectable/usable phenotype from appearing, and having a phenotype caused by reducing/eliminating the compensatingly differential expression of the gene or by labeling the compensatingly differentially regulated gene product.

Another aspect of the invention relates to the use of a genetically modified organism prepared according to the invention for screening for substances having an effect on the function of the heterologous protein or protein fragments and on a method for identifying substances having an effect on the function of the heterologous protein or protein fragment.

According to another aspect, the invention also relates to an assay for drug screening using a phenotyped organism of the invention by determining the phenotype (e.g. a growth inhibition due to induced heterologous overexpression of a protein), contacting the substance to be tested with said organism and observing a possible modification of said phenotype, preferably its at least partial reversion to the behavior or morphology of the wild-type organism (i.e. at least partial restoration of the phenotype of the starting organism, for example ending the growth inhibition). Furthermore, substances are concerned which are identified as being effective by a method of the invention or an assay of the invention.

The invention is illustrated in more detail below on the basis of examples.

### EXAMPLE 1

Development of a platform technology for identifying drugs which act on the activity of kinases, based on yeast as organism. The phenotype produced in this case is the growth inhibition of yeasts. The assay principle is thus based on the growth inhibition of yeasts which are used as living “reagent tube”. Growth inhibition here means, for example, a cell cycle arrest or lysis of the cells concerned. Yeasts are used, since they are ideally suited, owing to their genetic manipulability. Human (or other exogenous) kinases are overexpressed in the yeast and under the control of a galactose-inducible promoter (GAL1/10). The yeasts are transformed and cultured according to standard methods. Examples of vectors used are those of the p41x-GAL1 or p42x-GAL11 series.

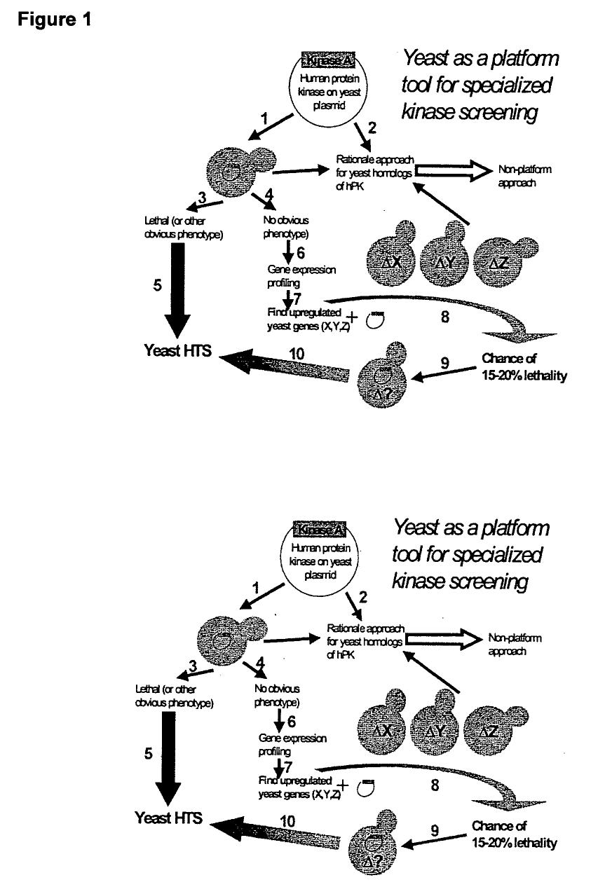

In approx. 30% of all kinases to be tested, overexpression will already result in growth inhibition in yeast (Tugendreich et al. (2001)). This procedure is documented in FIG. 1 by steps 1, 3, 5. Kinases whose overexpression results in growth inhibition are integrated into a suitable yeast strain and then transferred to high throughput screening (HTS). This example uses yeast strains of the strain background “MATa his3□1 leu2□0 met15□0 ura3□0” (BY4741 from EUROSCARF).

During assay development for the HTS, conditions are optimized by assaying various “drug transporter” deletion mutants in the above-described strain background. For all protein kinases to be tested in this example, the strains having the following deletion combinations are assayed: 1. YRWS21 (MATa pdr1Δ::KanMX pdr3Δ::KanMX his3Δ1 leu2Δ0 met15Δ0 lys2Δ0 ura3Δ0) 2. YRWS39 (MATa pdr5Δ::KanMX yor1Δ::KanMX his3Δ1 leu2Δ0 MET15 lys2Δ0 ura3Δ0) 3. YRWS14 (MATa pdr5Δ::KanMX snq2Δ::KanMX his3Δ1 leu2Δ0 MET15 lys2Δ0 ura3Δ0) 4. YRWS13 (MATa snq2⁻Δ::KanMX yor 1Δ::KanMX his3Δ1 leu2Δ0 MET15 lys2Δ0 ura3Δ0) 5. YRWS44 (MATa pdr5Δ::KanMX snq2Δ::KanMX yor1Δ::KanMX his3Δ1 leu2Δ0 met15Δ0 lys2Δ0 ura3Δ0).

It is then possible to search in high throughput screening for biological and chemical molecules which reduce or eliminate growth inhibition—i.e. which result in the growth of the yeast cultures. All previously described techniques are known to the competent skilled worker.

As described above, approx. 30% of all exogenous kinases cause growth inhibition in yeast. Therefore, approx. 70% of all overexpressed kinases cause only low, if any, growth inhibition. In order to utilize the principle of growth inhibition of yeast as platform technique for compound screening of all protein kinases, the remaining 70% of protein kinases must also cause growth inhibition. For this purpose, the present invention is needed.

The desired protein kinases are cloned into a yeast expression vector of choice, in this example p413 GAL1 (D. Mumberg et al. (1994) in full length and with a C-terminal tag, e.g. MYC tag). After transformation using the lithium acetate method according to a standard protocol (see Methods in Yeast Genetics) and culturing in a suitable medium, overexpression of the exogenous kinases in the yeast is induced by adding galactose according to a standard protocol (20 g/ml of medium) at 30° C. for 4 to 6 hours. Expression of the kinases is checked by immunoblots according to a standard protocol with the aid of antibodies against the chosen tag (e.g. anti-MYC: AB1364 (Chemikon) or M5546 (Sigma); anti-HA: HA-11-A (Biotrend) or 55138 (ICN)).

After the immunological detection of expression in the yeast, modifications in gene expression—caused by expression of the exogenous kinases—in the yeast (compensatingly differential regulation) are studied with the aid of DNA microarrays. DNA microarrays are support materials to which specific oligonucleotides are chemically coupled. The individual oligonucleotides here represent individual genes. DNA microarrays are used as tools which can cover the current expression pattern of the entire yeast genome. For this type of experiment, kinase-transformed yeasts are compared to mock-transformed (empty plasmid) yeasts as control. Total RNA is prepared from both strains by standard methods. The RNA is then hybridized with the chip-coupled oligonucleotides (on the microarrays) at 45° C. for 16 h. The direct comparison of the kinase-transformed yeast RNA with the mock-transformed yeast RNA reveals yeast genes which are regulated in a compensatingly differential manner by an overexpressed protein kinase. Studies of the inventors have shown that a genetic intervention, for example, in overexpression of an exogenous protein kinase, upregulates a particular number of RNAs for yeast genes and downregulates a particular number (table 1). This was carried out on the example of human kinase PAK1.

Table 1: 2 genes are upregulated, 11 genes are downregulated. Furthermore, the inventors were able to show for the first time, that many of the upregulated genes are upregulated for compensatory reasons. In this case, an S. cerevisiae wild-type strain (W303-1a (strain background or source of supply)) was compared with strain having a deletion in the Saccharomyces cerevisiae gene cla4 (□cla4) (YEL252). Apart from the deletion in the gene for CLA4, both strains are isogenic, i.e. identical. When comparing directly the RNA preparations from the two different strains (W303-1a and YEL252), 110 different RNAs of the yeast genome turned up as upregulated (table 2).

Table 2: 56 genes were downregulated (data not shown). Here, an increase of the RNA copy number for particular genes could possibly occur for compensatory reasons. In this specific example, compensatory means that the defect in the genetically modified strain, caused by deletion of the CLA4 gene, should be diminished by the increased expression of genes which can take over the function of CLA4 entirely or partially. In order to prove this thesis, some of the upregulated genes were selected for further experiments (see “2nd deletion” in table 3).

Table 3: For this purpose, MATE□ yeast strains (which may be obtained, for example, from EUROSCARF or Research Genetics) were selected which carry deletions in the in each case upregulated genes. The deletions are marked by marker genes, i.e. marker genes, for example, for an antibiotic resistance or for required growth factors such as, for example, particular amino acids are integrated into the particular yeast genome. The deletion strains selected were crossed with the CLA4 deletion strain (YEL252, MATa) according to standard methods of yeast genetics (Methods in Yeast Genetics: A Cold Spring Harbor Course Manual (1994)).

After crossing, diploid yeasts were selected which were then induced to form spores. This involves generation of 4 haploid spores from a diploid yeast cell, which can be divided into 4 haploid yeast clones for germination. Accordingly, the genes of the diploid strain become newly distributed. In 25% of all cases, the 2 deletions of the different starting strains will be united in a new haploid clone. This may be readily monitored on the basis of the various selection markers.

This standard method was used to try to prepare 13 different double deletions. In only 10 cases, the double deletions were viable, in 3 cases, the double deletion never took place (table 3 “lethal”). In all 3 cases, 40 asci were tested. It is therefore clear that the combination of both deletions causes the affected spore to die. They are also synthetically lethal. It was demonstrated that in all 13 cases the double deletions were either synthetically lethal or have displayed other synthetic phenotypes (table 3). This study confirms the thesis that the affected genes were upregulated in order to compensate for defects caused by the lack of CLA4. It is important to the invention that in the cases studied (13 double deletions) 3 combinations and thus 23% of all possible double deletions displayed synthetic lethality (table 3).

In the experiment with the □cla4 strain, 110 genes were upregulated (table 2). In the same way, overexpression of human PAK1 in the above-described approach upregulated the mRNAs of 2 genes (table 1). Consequently, these genes are also upregulated for compensatory reasons. Owing to the small number of upregulated genes and the low rate of success connected therewith for synthetically lethal combinations, we dispensed with the follow-up experiment of identifying strains which displayed a synthetically lethal phenotype in the combination of deletions in the upregulated genes (with YMR096W or HIS3 of table 1) and expression of human PAK1. Rather, a hyperactive mutant of human PAK1 was produced, namely human PAK1□CRIB. This mutant was transformed into yeast, again using standard methods. Owing to the high kinase activity, this protein caused growth inhibition in the yeast. A suitable strain for assaying low-molecular weight substances had been identified. The goal had been achieved. Nevertheless, in this case too, a differential expression profile was recorded using the DNA microarrays, in order to back up the validity of the invention (table 4).

Table 4: 55 different yeast genes were compensatingly upregulated, owing to the high kinase activity, and 3 genes were downregulated (not shown). If the high activity of the PAK mutant had not been sufficient to cause growth inhibition in the yeast, it would now be possible to assay deletion strains for the upregulated genes. The PAK1 mutant would have to be expressed in the particular deletion strain. On the basis of the value of a 23% chance of success in a synthetic phenotype, expression of the human PAK1 mutant would then cause growth inhibition in approx. 13 yeast strains. Thus a strain for assaying potential kinase inhibitors would have been identified.

In the case of assaying human kinases in the yeast, the starting strains would not need to be crossed, since the human kinases is expressed from a plasmid in a galactose-dependant manner. Said plasmid need only be transformed into the particular deletion strain and expression of the kinase needs to be induced. In 23% of all cases of the strains to be assayed, it will be possible to observe growth inhibition (lethality). The growth-inhibited strains can no longer compensate expression of the plasmid-encoded protein kinase, owing to the particular deletions. Therefore, these systems can be transferred to HTS.

Should overexpression of particular wild-type kinases in combination with the DNA-microarray experiment not be sufficient (as described above for wild-type PAK1, see table 2) to cause growth inhibition, then mutants of the particular kinase are prepared and used instead of said wild-type kinases (also for the gene expression experiments using the DNA microarrays). These mutants may be prepared according to the principle of random mutagenesis, with the aim of obtaining hyperactive mutants. For mutagenesis, the kinase constructs are used with a C-terminal tag according to the method of Tugendreich et al. (2001).

Thus, for the first time and surprisingly, studies of the inventors showed that the deletion of compensatingly differentially regulated genes can result in growth inhibition and in the finding connected therewith of designing a standardized platform assay for protein kinases. In the actual experiments, growth inhibition was detected with a frequency of 23%. The deletion strains which exhibit growth inhibition after transformation with the plasmid-encoded protein kinase may then, as described above, be transferred to HTS by means of optimization (testing of the various drug-transporter knockouts). FIG. 1 illustrates the invention by way of example on the basis of points 1, 4, 6-10.

Apart from crossing-in the deletions of compensatingly differentially regulated genes, deletion thereof could also have been carried out using other methods such as genomic knockout of the kinase-expressing yeast itself. However, in yeasts the elimination of compensatingly differentially regulated genes by crossing in deletions or the genomic knockout is particularly advantageous, owing to the simplicity of the procedure. In contrast, other methods may be more suitable in other organisms. Thus, in the example of eukaryotic cell lines and in the case of multicellular organisms such as Drosophila and C. elegans, the application of antisense methods such as RNAi is more suitable. The selection of measures suitable in each case for the individual organisms is within the ability of the skilled worker.

The platform assay of the invention enables HTS of all protein kinases (as described on the basis of human PAK1) in homogeneous and thus cost-effective assay systems. This system is also suitable for determining IC₅₀ values in compound screening.

As described in the example, the gene expression experiments also result in the identification of RNAs of genes which are repressed by expression of exogenous kinases. The promoters of said repressed genes may serve as reporters in HTS. For this purpose, the yeast promoters are fused to “reporter genes” such as β-galactosidase, luciferase, growth markers such as HIS3, URA3, LEU2, or TRP1, etc. These constructs are transformed into the yeast strain for HTS. There they serve as growth markers for compounds which eliminate growth inhibition in the affected strain.

### EXAMPLE 2

The platform assay may also be used as “multiplex system”. Multiplex system means assaying various proteins or protein fragments, for example kinases, in the same assay in one reaction mixture at the same time. For this purpose, the individual phenotyped yeast strains are constructed first. The exogenous protein kinases are integrated using standard methods (see above). These yeast strains are then mixed to give a homogeneous culture. Expression of the protein kinases in the homogeneous yeast strain mixture results in growth inhibition, since expression of each individual kinase per se causes growth inhibition in the phenotyped yeast strain. HTS identifies compounds which result in the growth of at least one yeast strain. It is then essential to assign the kinase concerned to said compounds. This is achieved via the “colony PCR” method (A. J. P. Brown and M. Tuite (1998)). For this purpose, a few microliters from the growing yeast cultures are lysed, following instructions (A. J. P. Brown and M. Tuite (1998)). Quantitative RT-PCR using specific primers for the different protein kinases identifies unambiguously the inhibited kinase(s) concerned from (the mixture of) genomic DNA (including integrated protein kinases). Thus it is possible to assay in a single screening different kinases by mixing equal parts of different yeast strains. The advantage is an enormous saving of cost and time.

This technology is applicable not only to protein kinases but to any proteins or substances which cause a transcriptional response in the yeast.

This platform assay enables in the subject to assays of the prior art, for example, HTS of all protein kinases (not only of those whose heterologous expression already produces a phenotype immediately) in homogeneous and therefore cost-effective assay systems. This system is also suitable for determining IC₅₀ values in compound screening.

This technology is applicable not only to protein kinases but to all proteins or substances which cause a transcriptional response in the yeast.

Methods:

The standard methods according to Sambrook et al. (1989) Molecular Cloning: A Laboratory Manual, Second edition, Cold Spring Harbor Laboratory Press, Cold Spring Harbor, N.Y. 545 pp. were used for genetic manipulations.

Growth conditions, crossing conditions and genetic manipulations on yeasts (Saccharomyces cerevisiae) were carried out according to Guthrie, C. and G. R. Fink (1991) Guide to Yeast Genetics and Molecular Biology, Volume 194, J. N. Abelson and M. I. Simon, eds. (San Diego, Calif.: Academic Press Inc.). The Affymetrix experiments (“gene expression analysis) were carried out exactly according to Klebl et al. (2001) Biochem. Biophys. Res. Commun. 286, 714-720.

References

Brown, A. J. P. and M. Tuite (1998), PCR-Based Gene Targeting in Saccharomyces cerevisiae. Methods Microbiol. 26, 67-81.

Methods in Yeast Genetics; A Cold Spring Harbor Course Manual; 1994 Edition; Kaiser, C., Michaelis, S., and A. Mitchell; Cold Spring Harbor Laboratory Press.

Mumberg, D., Müller, R. and M. Funk (1994). Regulatable promoters of Saccharomyces cerevisiae: comparison of transcriptional activity and their use for heterologous expresson. Nucl. Acids Res. 22, 5767-5768.

Tugendreich, S., Perkins, E., Couto, J., Barthmaier, P., Sun, D., Tang, S., Tulac, S., Nguyen, A., Yeh, E., Mays, A., Wallace, E., Lila, T., Shivak, D., Prichard, M., Andrejka, L., Kim. R. and T. Melese (2001). A streamlined process to phenotypically profile heterologous cDNAs in parallel using yeast cell-based assays. Genome Res. 11, 1899-1912.

TABLE 1
2 genes are upregulated in the ste20Δ strain YEL206
which expresses hPAK1

| | | x-fold |
|---|---|---|
| Remarks | Gene function | upregulated |
| YMR096W | Stationary phase protein | 2.15 |
| HIS3 | Imidazole glycerol phosphate dehydratase; | 6.77 |
| | 7th step of histidine biosynthesis | |

11 genes are downregulated in the ste20Δ strain YEL206 which
expresses hPAK1

| | | x-fold |
|---|---|---|
| Remarks | Gene function | downregulated |
| STE20 | Serine/threonine protein kinase of the | 47.62 |
| | pheromone response signal | |
| | transduction pathway | |
| FRE7 | Protein with weak similarity to Fre1p | 11.70 |
| | and Fre2p, involved in iron transport | |
| MFA1 | Mating pheromone a-factor, exported | 3.70 |
| | from the cell by Ste6p | |
| YLR042C | Unknown | 3.27 |
| GPH1 | Glycogen phosphorylase, releases α-D- | 2.63 |
| | glucose 1-phosphate | |
| FRE1 | Iron and copper reductase, acts on | 2.55 |
| | Fe2+ ion chelates | |
| YHR087W | Unknown | 2.31 |
| CWP1 | Cell wall mannoprotein; member of | 2.27 |
| | the PAU1 family | |
| YJL217W | Unknown | 2.25 |
| CTR1 | Copper transport protein; required for | 2.17 |
| | high-affinity uptake of copper ions; | |
| FET4 | Low-affinity Fe(II) transport protein | 2.00 |

TABLE 2
110 genes are upregulated in the cla4□ strain YEL 252

| | Remarks | Gene function | x-fold upregulated |
|---|---|---|---|
| Cell wall | FKS2 | Component of □-1,3-glucan synthase, | 6.81 |
| maintenance | | probably functions as alternative subunit to | |
| | | Fks1p (88% identical); 55% identical to | |
| | | Fks3p; interacts with Rho1p; | |
| | | fks1Δfks2Δ is lethal | |
| | ECM29 | Possibly involved in cell wall structure or | 3.13 |
| | | biosynthesis | |
| | SPI1 | Bound to cell wall via GPI anchor; induced | 2.72 |
| | | by Msn2/4p | |
| | SBE22 | Required for growth of buds; involved in cell | 2.08 |
| | | wall integrity | |
| Cellular | HSP12 | 12 kDa heat shock protein, induced by heat, | 6.55 |
| stress | | osmotic (HOG1-, PBS2-dependent) or | |
| | | oxidative stress, stationary phase, HSF1, | |
| | | MSN2, YAP1; chaperone (member of the | |
| | | hydrophilin family); 5 STREs | |
| | HSP26 | Heat shock protein, induced by osmotic | 4.76 |
| | | stress, HSF1, MSN2, heat, H₂O₂; 29% | |
| | | identical to Hsp42p; chaperone; 4 STREs | |
| | HSP82 | Heat shock protein, 97% identical to | 2.67 |
| | | Hsc82p, similar to mammalian HSP90 | |
| | | (complementable by human HSP90); | |
| | | chaperone; induced by HSF1, SKN7, YAP1, | |
| | | H₂O₂; has ATPase activity; partly regulated | |
| | | by HOG1 signal pathway, binds to Ste11p; | |
| | | HSP90 activity is modulated by Sch9p | |
| | GPX2 | Glutathione peroxidase, induced by YAP1 & | 2.64 |
| | | oxidants | |
| | SKN7 | Transcription factor, involved in response to | 2.60 |
| | | oxidative stress (H₂O₂) and G1 cell cycle | |
| | | control (appearing of buds); interacts with | |
| | | Rho1p, Mbp1p, Cdc42p & genetically with | |
| | | PKC1; required for N₂-withdrawal-induced | |
| | | pseudohyphal growth; cooperates with | |
| | | Yap1p in induction of gene expression; not | |
| | | involved in heat shock; possibly participates | |
| | | in HOG1 signal pathway; part of a two- | |
| | | component system; transcription activation | |
| | | stimulated by skn7p depends on Ras/PKA | |
| | | signal pathway | |
| | SOD2 | Mitochondrial Mn2+ superoxide dismutase, | 2.57 |
| | | induced by HAP1, 2, 3, 4, 5 & repressed by | |
| | | cAMP (RAS2); transcriptional response to | |
| | | H₂O₂ is Yap1p- & Skn7p-dependent; | |
| | | induced by Msn2/4p | |
| | ICT1 | k.o. higher resistance to Cu2+ than wild | 2.41 |
| | | type; mitochondrial energy transfer | |
| | | signature | |
| | CYP2 | Member of cyclophilin family, heat shock | 2.37 |
| | | protein, isomerase, chaperone | |
| | HSP42 | Heat shock protein, involved in restoration | 2.28 |
| | | of cytoskeleton during mild stress effect; | |
| | | induced by HOG1, MSN2/4, EtOH, H₂O₂; 3 | |
| | | STREs | |
| | MSN4 | Strong similarity to Msn2p; regulation of | 2.15 |
| | | trehalose concentration during stress; 39 | |
| | | genes dependent on Msn2/4p for induction | |
| | | in diauxic shift and repressed by cAMP; | |
| | | ALD3, GDH3, GLK1, HOR2, HSP104, | |
| | | HXK1, PGM2, SOD2, SSA3, SSA4, TKL2, | |
| | | TPS1, ARA, e.g. Ras2p controls stress | |
| | | response gene expression by Msn2/4p & | |
| | | Yap1p; TOR signal transduction controls | |
| | | nuclear localization of nutrient-regulated | |
| | | transcription factors | |
| Nucleotide | ADE2 | Phosphoribosylaminoamidazole | 5.96 |
| metabolism | | carboxylase (AIR decarboxylase); white vs | |
| | | red colonies | |
| | ADE17 | 5-Aminoimidazole-4-carboxamide | 3.42 |
| | | ribonucleotide (AICAR) transformylase/IMP | |
| | | cyclohydrolase; white vs red colonies | |
| | DCD1 | Deoxycyticylate deaminase; k.o. has | 2.50 |
| | | increased dCTP pool | |
| Transport of | FRE7 | Involved in uptake of copper and iron; weak | 4.98 |
| small | | similarity to Fre1p | |
| molecules | | | |
| | YHR048W | 29% identical to Ygr138p, Ypr156p, and | 4.20 |
| | | 33% to Flr1p; MFS-MDR member | |
| | PHO89 | High-affinity Na+-dependent phosphate | 2.76 |
| | | transporter; | |
| | YGR138c | Member of the cluster I (family 1) of the | 2.54 |
| | | MFS-MDR 89% identical to Ypr156p | |
| | YER053C | MCF member | 2.40 |
| | TAF1 | Triacetylfuscerinine C transporter (MDR- | 2.24 |
| | | MFS); 56%, 46%, 46% identical to Arn1p, | |
| | | Ycl073p, Ykr106p | |
| | MUP3 | Low affinity amino acid permease (Met | 2.16 |
| | | permease); APC family member | |
| | ATM1 | ABC superfamily member, required for | 2.03 |
| | | growth; may function in sensing iron; 43% | |
| | | identical to human ABC7 | |
| Carbohydrate | GRE3 | NADPH-specific aldose reductase, induced | 3.61 |
| metabolism | | by osmotic stress, MSN2/4, 0.1 M LiCl; 36%, | |
| | | 34%, 34% identical to Yjr096p, Gcy1p, | |
| | | Ypr1p; STREs and PDSEs; similar to | |
| | | human 305B protein (neonatal cholestatic | |
| | | hepatitis) | |
| | GPH1 | Glycogen phosphorylase repressed by | 3.49 |
| | | cAMP; stress-inducible | |
| | GUT1 | Glycerol kinase, catalyzes conversion of | 3.37 |
| | | glycerol to glycerol-3-phosphate, induced by | |
| | | ADR1, INO2, INO4, glycerol; strong | |
| | | similarity to human GK; activity is reduced | |
| | | during osmotic stress | |
| | PCY1 | Pyruvate carboxylase l; converts pyruvate | 2.50 |
| | | to oxalacetate for gluconeogenesis; 93%, | |
| | | 30%, 38% identical to Pyc2p, Hfa1p, | |
| | | Dur1,2p; similar to human PYC | |
| | TSL1 | Component of trehalose-6-phosphate | 2.40 |
| | | synthase/phosphatase complex; induced by | |
| | | STE12, STE7, TEC1, osmotic stress & | |
| | | repressed by cAMP, glucose; contains | |
| | | STREs | |
| | GLK1 | Glucokinase specific dor aldohexoses; 73%, | 2.09 |
| | | 38%, 37% identical to Ydr516p, Hxk1p, | |
| | | Hxx2p; induced by GCR1, HOG1, MSN2, | |
| | | MSN4 & repressed by cAMP, cold; protein | |
| | | increased upon H₂O₂, G1 phase | |
| Protein | YPS3 | GPI-anchored aspartyl protease (yapsin) at | 3.40 |
| degradation | | the plasma membrane; 45%, 36%, 47% | |
| | | identical to Mkc7p, Sst1p, Yps1p | |
| | UB14 | Ubiquitin polyprotein, mature ubiquitin is | 3.27 |
| | | cleaved from polyubiquitin (Ubi4p) or from | |
| | | fusions with ribosomal proteins Rps31p, | |
| | | Rp140Ap, Rp140Bp; ribosomal heat shock | |
| | | protein & protein conjugation factor; 90% | |
| | | identical to Rpl40A/Bp and 100% to | |
| | | Rps31p; induced HSF1, MSN2, starvation, | |
| | | heat shock; required for survival of cell | |
| | | stress; k.o. is hypersensitive to H₂O₂, N₂- | |
| | | and C₂-starvation; has STREs and HSEs | |
| | VID24 | Required for vacuolar import and | 2.82 |
| | | degradation of Fbp1p | |
| | RPN10 | Non-ATPase component of the 26S | 2.46 |
| | | proteasome complex, binds ubiquitin- | |
| | | lysozyme conjugates in vitro; C-terminus | |
| | | binds to ubiquitin | |
| | BUL1 | Involved in ubiquitination pathway, binds to | 2.12 |
| | | ubiquitin ligase | |
| | AAP1 | Ala/Arg aminopeptidase, related to other | 2.00 |
| | | Zn2+ metalloproteases & mammalian Zn2+ | |
| | | aminopeptidases | |
| DNA | RIM1 | Transcription factor which binds ssDNA; | 3.27 |
| synthesis | | required for replication in mitochondria | |
| Amino acid | YMR250W | Similar to glutamate decarboxylase | 3.11 |
| metabolism | | | |
| | GDH2 | Glutamate DH, primary pathway to generate | 2.83 |
| | | NH₄⁺ from glutamate, induced by | |
| | | rapamycin; gets phosphorylated in | |
| | | response to N₂ starvation (inactivation; | |
| | | PAK-dependent) | |
| | GCV1 | Glycine decarboxylase T subunit, functions | 2.31 |
| | | in pathway for Gly degradation | |
| | CHA1 | Mitochondrial L-Ser/L-Thr deaminase, | 2.17 |
| | | catalyzes conversion of Ser to pyruvate & | |
| | | Thr to □-ketobutyrate; induced by Ser, Thr, | |
| | | SIL1, CHA4 | |
| Signal | YGL179C | Ser/Thr protein kinase with similarity to | 3.10 |
| transduction | | Elm1p (31%), Pak1p (49%), Kin82p (30%), | |
| | | Gin4p (29%) | |
| | KSP1 | Ser/Thr protein kinase that suppresses | 2.85 |
| | | prp20Δ when overexpressed | |
| | SLT2 | Ser/Thr protein kinase of the MAP kinase | 2.77 |
| | | family involved in the cell wall integrity | |
| | | pathway, polarized growth, response to | |
| | | nutrient availability, heat shock; interacts | |
| | | with Rlm1p, Swi4/6p, Mkk1/2p, Spa2p, | |
| | | Ptp2/3p, phosphorylates Swi4/6p & | |
| | | functions as regulator of the SBF complex; | |
| | | kinase activity induced by pheromone | |
| | | (requires Ste20p, but not Ste12p); kinase | |
| | | activity is cell cycle regulated | |
| | STE20 | Ser/Thr protein kinase of pheromone | 2.25 |
| | | response pathway, participates also in | |
| | | filamentous growth and STE vegetative | |
| | | growth pathways; | |
| | YCK1 | CKI isoform, 77%, 50%, 41% identical to | 2.21 |
| | | Yck2p, Yck3p, Hrr25p and 50-55% with | |
| | | human isoforms; gernaylgeranylated; | |
| | | yck1Δyck^{ts} displays hyperpolarized growth, | |
| | | hypersensitivity towards Zn²⁺ and multiple | |
| | | drugs, resistance to Mn²⁺ | |
| | YHR046C | Myo-inositol-1 (or -4)-monophosphatase, | 2.17 |
| | | participates in inositol cycle of Ca²⁺ signalng | |
| | | & inositol biosynthesis; similar to human | |
| | | MYOP (anti-manic, and -depressive actions | |
| | | of Li⁺) | |
| | SCH9 | Ser/Thr protein kinase activated by cAMP; | 2.17 |
| | | 46%, 44%, 42% identical to Ypk2p, Ypk1p, | |
| | | Tpk3p & 49% to human AKT1,2; controls | |
| | | FGM pathway; k.o. has modest defect in | |
| | | pseudohyphal growth and displays | |
| | | hyperinvasive growth | |
| | PTP2 | PTPase involved in Hog1p and pheromone | 2.01 |
| | | response pathways; interacts with Hog1p, | |
| | | Slt2p; induced by SLT2, YAP1, heat, | |
| | | osmotic stress; dephosphorylates Hog1p, | |
| | | Fus3p; posttranslationally regulated by | |
| | | Hog1p; 2 STREs | |
| Lipid, fatty | PLB3 | Phospholipase B, releases GPI into the | 3.01 |
| acid & sterol | | medium | |
| metabolism | | | |
| | ERG7 | Lanosterol synthase (ergosterol | 2.30 |
| | | biosynthesis), essential | |
| Membrane | YHR138C | Involved in vacuolar fusion with sequence | 2.81 |
| fusion | | similarity to Pbi2p | |
| Cell cycle | PCL5 | Cyclin that associates with Pho85p, belongs | 2.73 |
| control | | to Pcl1/2p subfamily | |
| Polll | GAT2 | GATA Zn²⁺ finger transcription factor, | 2.73 |
| transcription | | required for expression of N₂ catabolite | |
| | | represson-sensitive genes | |
| | HAP4 | Transcription factor, component of the | 2.48 |
| | | Hap2/3/4/5p-complex involved in activation | |
| | | of CCAAT box-containing genes (SOD2, | |
| | | e.g.) | |
| | STP4 | Transcription factor with strong homology to | 2.17 |
| | | Stp1, 2, 3p; involved in tRNA splicing and | |
| | | branched-chain amino acid uptake | |
| | SNF6 | Transcription factor, component of the SWI- | 2.13 |
| | | SNF global transcription activator complex; | |
| | | acidic domains of Gcn4p, Swi5p, Hap4p | |
| | | interact directly with SWI-SNF complex | |
| | SET1 | Transcription factor of the trithorax family of | 2.04 |
| | | SET-domain-containing proteins, | |
| | | participates in control of transcription and | |
| | | chromosome structure; similar to human | |
| | | HRX Zn²⁺ finger protein | |
| Energy | MDH2 | Cytosolic malate DH (glyoxylate cycle); | 2.60 |
| generation | | induced by N₂ source limitation & repressed | |
| | | by cAMP, glucose; 3 STREs | |
| RNA | RPP1 | Subunit of ribonuclease P & Rnase MRP | 2.49 |
| processing/ | | ribonucleoprotein particles, needed for | |
| modification | | tRNA & 5.8S rRNA processing; 23% | |
| | | identical to hRpp30 | |
| | PRP8 | U5 snRNA-associated splicing factor; | 2.41 |
| | | essential RNA-binding protein; 62% | |
| | | identical to human PRP8; component of the | |
| | | spliceosome | |
| | RRP4 | 3′-5′-exoribonuclease required for 3′- | 2.38 |
| | | processing of ribosomal 5.8S rRNA; | |
| | | component of the nuclear & cytoplasmid | |
| | | forms of the 3′-5′-exosome complex; | |
| | | essential; induced in S-phase | |
| | DBP8 | Similar to DEAD box family of RNA | 2.33 |
| | | helicases | |
| Other | YNL274C | Potential □-ketoisocaproate reductase, | 2.26 |
| metabolism | | induced by YAP1, H₂O₂ | |
| | DUR1, 2 | Urea amidolyase, contains urea caroxylase | 2.21 |
| | | & allophanate hydrolase activities; | |
| | | repressed by NH₄⁺ & induced by N₂ | |
| | | starvation, mating pheromone, Arg, | |
| | | rapamycin (N₂ utilization gene) | |
| Protein | UBP5 | Ubiquitin-specific protease homologous to | 2.17 |
| modification | | Doa4p & human Tre-2; member of | |
| | | rhodanese homology family | |
| Protein | MSR1 | Mitochondrial arginyl-tRNA synthetase, 61% | 2.17 |
| synthesis | | identical to Ydr341p | |
| Vesicular | SFB3 | Possible component of COPII vesicles, | 2.17 |
| transport | | involved in transport of Pma1p from eR to | |
| | | Golgi; interacts with Sec23p | |
| Cytokinesis | CDC12 | Essential part of the septin complex at the | 2.09 |
| | | neck; required for pheromone-induced | |
| | | morphogenesis; septin assembly depends | |
| | | on Cla4p & Ste20p (Cdc42p, Cdc24p); | |
| | | mislocalized in yck2^{ts} | |
| Mating | SSF1 | Suppressor of sterile four; 94% identical to | 2.06 |
| response | | Ssf2p; ssf1□ssf2□ is lethal; multicopy | |
| | | suppressor of hsp90-loss-of-function | |
| | | mutation | |
| Unknown | YHR214W | 100%, 77%, 74% identical to Yar066p, | 9.88 |
| | | Yil169p, Yol155p | |
| | YAR066W | 100%, 77%, 74% identical to Yhr214p, | 7.59 |
| | | Yil169p, Yol155p | |
| | RTA1 | Resistant to aminocholesterol; induced by | 4.64 |
| | | TEC1, STE7, STE12 | |
| | MSC1 | Functions in the meiotic homologous | 4.62 |
| | | chromatid recombination pathway | |
| | YHL021C | Induced by STE12, TEC1, STE7 | 4.35 |
| | YHR209W | Putative SAM-dependent methyltransferase | 4.26 |
| | COS8 | Protein family of conserved sequences | 3.74 |
| | YNR014W | 30% identical to Ymr206p; 4 putative | 3.44 |
| | | STREs | |
| | YIR042C | — | 3.37 |
| | YCL049C | — | 3.28 |
| | YHR087W | — | 3.19 |
| | YHR078W | 4 potential transmembrane segments | 3.00 |
| | TRA1 | Essential component of the Ada-Spt | 2.82 |
| | | transcriptional regulatory complex (SAGA), | |
| | | SAGA-like complex, & NuA4 complex | |
| | BTN2 | Elevated expression with yhc3Δ; 38% | 2.77 |
| | | identical to human HOOK1 | |
| | VAB36 | Vac8p-binding protein of 36 kDa; 2 putative | 2.75 |
| | | STREs | |
| | YFL063W | Similar to subtelomeric proteins | 2.68 |
| | YHR112C | Similar to cystathione □-synthase Str2p & | 2.56 |
| | | other transulfuration enzymes, also similar | |
| | | to human CGL (cystathioninuria) | |
| | YBL064C | Mitochondrial thiol peroxidase of the 1-Cys | 2.55 |
| | | family; one of the 4 peroxidases in S.c.; | |
| | | uses thioredoxin as electron donor; induced | |
| | | upon oxidative stress; reduces H₂O₂ in the | |
| | | presence of DTT | |
| | YSC83 | Induced mRNA levels during sporulation | 2.46 |
| | BOP1 | Bypass of PAM1 (PAM1 = multicopy | 2.45 |
| | | suppressor of loss of PP2A) | |
| | YHR045W | 5 potential transmembrane domains | 2.44 |
| | YHR033W | Induced by N₂ source limitation & repressed | 2.42 |
| | | by cAMP | |
| | YPR009W | Putative Zn²⁺-finger domain; 34% identical | 2.40 |
| | | to Sut1p | |
| | YLL064C | Member of the seripauperin family | 2.39 |
| | YPL137C | Similar to Mhp1p (27%), Yor227p (43%) | 2.39 |
| | YHR182W | — | 2.37 |
| | YDR222W | — | 2.37 |
| | YHR146W | Similar to pheromone adaption protein | 2.36 |
| | | Mdg1p | |
| | YMR184W | — | 2.36 |
| | YGL261C | Member of the seripauperin (PAU) family | 2.34 |
| | YHR083W | Essential | 2.32 |
| | YHR122W | Essential | 2.29 |
| | YOR227W | 43%, 25% identical to Ypl137p, Mhp1p | 2.27 |
| | YHR186C | WD40 domain; essential | 2.26 |
| | YHR073W | Similar to human oxysterol-binding protein; | 2.20 |
| | | interacts with Spo12p | |
| | YJL217W | — | 2.17 |
| | YHR192W | — | 2.11 |
| | YDL231C | Putative Zn²⁺ finger domain | 2.10 |
| | YDR391C | 57%, 41% identical to Yor013p, Yor012p | 2.05 |

TABLE 3

| Name | 1st | 2nd | |
|---|---|---|---|
| of strain | deletion | deletion | Phenotype |
| W303-1a | — | — | none |
| YEL252-1a | cla4 | — | cytokinesis |
| YAS | cla4 | ptp2 | synthetic |
| YAS | cla4 | glk1 | synthetic |
| YAS | cla4 | msn4 | synthetic |
| YAS | cla4 | ygl173 | synthetic |
| YAS | cla4 | gut1 | synthetic |
| YAS | cla4 | rta1 | cured |
| YAS | cla4 | skn7 | synthetic |
| YAS | cla4 | pde2 | synthetic |
| YAS | cla4 | yck1 | synthetic, extremely |
| | | | slow growth |
| YAS | cla4 | sbe22 | synthetic |
| YAS | cla4 | elm1 | lethal |
| YAS | cla4 | slt2 | lethal |
| YAS | cla4 | ste20 | lethal |

TABLE 4
55 genes are upregulated in the ste20Δ strain YEL206
which expresses hPAK1ΔCRIB

| | | x-fold |
|---|---|---|
| Remarks | Gene function | upregulated |
| PHO5 | Repressible acidic phosphatase; requires | 10.19 |
| | glycosylation for activity | |
| ZRT1 | High-affinity zinc transport protein; member | 10.12 |
| | of ZIP family | |
| PHO11 | Secreted acidic phosphatase | 7.67 |
| HSP30 | Heat shock protein, located in | 6.30 |
| | plasma membrane | |
| PHO12 | Secreted acidic phosphatase | 5.80 |
| YIL057C | Unknown | 5.70 |
| YOL154W | Protein with similarity to | 5.24 |
| | zinc metalloproteinases | |
| YPL274W | High-affinity 5-adenosylmethionine | 5.16 |
| | permease | |
| CIT3 | Mitochondrial citrate synthase | 5.15 |
| RTA1 | Protein involved in 7-aminocholesterol | 5.14 |
| | resistance | |
| YEL070W | Protein with similarity to E. coli | 5.09 |
| | D-mannonate oxidoreductase | |
| YDL037C | Protein with similarity to glucan | 4.95 |
| | 1,4-□-glucosidase | |
| YHR136C | Putative inhibitor of Pho80-Pho85p | 4.84 |
| | cyclin-dependent kinase complex | |
| LEE1 | Unknown | 4.59 |
| YMR303C | Alcohol dehydrogenase II; oxidizes | 4.07 |
| | ethanol to acetaldehyde | |

1. A method for generating a genetically modified organism for drug screening, which comprises the steps
a) causing heterologous expression of at least one protein or protein fragment by genetic modification of the organism
b) analyzing the modified gene expression pattern and identifying compensatingly differentially regulated genes
c) phenotyping the organism.

2. The method as claimed in claim 1, wherein phenotyping is carried out by reducing/eliminating the compensatingly differential expression or by labeling at least one compensatingly differentially regulated gene.

3. The method as claimed in either of claims 1 and 2, wherein the genetic modification causes heterologous expression of at least one protein or protein fragment which is endogenous to the organism and/or foreign.

4. The method as claimed in any of claims 1 to 3, wherein the genetic modification causes reduction or elimination of the expression of at least one protein endogenous to the organism.

5. The method as claimed in any of claims 1 to 4, wherein the modified expression is inducible.

6. The method as claimed in claim 5, wherein the genetic modification comprises introducing a vector which enables the protein or protein fragment to be inducibly expressed, preferably a vector inducible with galactose, copper tetracycline or other comparably inducible vectors.

7. The method as claimed in any of claims 1 to 6, wherein the genetic modification comprises a knock out, preferably an inducible knock out.

8. The method as claimed in any of claims 1 to 7, wherein the organism is drosophila, C. elegans, a prokaryotic or a eukaryotic cell.

9. The method as claimed in claim 8, wherein the cell is a yeast cell, preferably a yeast cell of the strain S. cerevisiae.

10. The method as claimed in any of claims 1 to 9, wherein the modified gene expression is analyzed with the aid of DNA or protein microarrays.

11. The method as claimed in any of claims 1 to 10, wherein pheno-typing is carried out by reducing or eliminating expression of the compensatingly differentially regulated gene.

12. The method as claimed in claim 11, wherein expression of the compensatingly differentially expressed gene is enhanced to control organisms and the reduction or elimination is caused by at least partial inhibition of said enhanced expression.

13. The method as claimed in claim 7, wherein the knock out of the differentially expressed gene is carried out [lacuna] replacing at least part of the coding sequence of the differentially regulated gene with the coding sequence of a reporter gene or parts of the reporter gene sequence which are sufficient to be detected.

14. The method as claimed in claim 11, wherein the differentially expressed gene is less strongly expressed than in control organisms and the reduction or elimination is carried out by enhancing its expression.

15. The method as claimed in any of claims 1 to 14, wherein the reduction or elimination leads to growth inhibition of the organism.

16. The method as claimed in any of claims 1 to 10, wherein phenotyping is carried out by labeling the gene product of the compensatingly differentially regulated gene.

17. A genetically modified, phenotype organism, obtained by a method as claimed in any of claims 1 to 16.

18. A genetically modified organism, having
a) genetically modified expression of at least one endogenous or foreign gene, which results in compensatingly differential expression of at least one other gene endogenous to said organism, and
b) a phenotype caused by reducing/eliminating the compensatingly differential expression of the gene or by labeling the compensatingly differentially regulated gene product.

19. The use of a genetically modified organism as claimed in either of claims 17 or 18 for screening for substances having an effect on the function of the heterologous protein or protein fragment.

20. A method for identifying substances having an effect on the function of the heterologously expressed protein or protein fragment, which method comprises the use of the organism as claimed in either of claims 17 or 18.

21. An assay for drug screening using at least one phenotype organism as claimed in either of claims 17 or 18, which comprises the steps
c) determining the phenotype of said organism
d) contacting the substance to be tested with said organism
e) observing a possible modification of said phenotype.

22. A substance, which is identified by a method as claimed in claim 20 or an assay as claimed in claim 21 as a substance which at least reduces the phenotype.
